(19) United States
(12) Patent Application Publication
(10) Pub. No.: US 2026/0094729 A1
(43) Pub. Date: Apr. 2, 2026

(54) MEDICAL INFORMATION PROCESSING DEVICE, MEDICAL INFORMATION PROCESSING METHOD, AND STORAGE MEDIUM

(71) Applicant: CANON MEDICAL SYSTEMS CORPORATION, Otawara-shi (JP)

(72) Inventor: Tomoki OHYASU, Sakura (JP)

(73) Assignee: CANON MEDICAL SYSTEMS CORPORATION, Otawara-shi (JP)

(21) Appl. No.: 19/339,986

(22) Filed: Sep. 25, 2025

(30) Foreign Application Priority Data
Sep. 30, 2024 (JP) 2024-171204

(51) Int. Cl. G16H 80/00; G16H 10/60

(52) U.S. Cl. CPC G16H 80/00; G16H 10/60

(57) ABSTRACT

A medical information processing device of an embodiment is a medical information processing device that receives a question for a patient from a medical professional and answers on behalf of the patient using a digital twin that reproduces the condition of the patient, the digital twin being an AI model trained by adding information on the patient to at least a large language model, the medical information processing device including processing circuitry. The processing circuitry receives the question, acquires at least medical condition information that represents the medical condition of the patient, updates the digital twin based on the medical condition information, uses the digital twin to output answer information representing the answer to the received question, generates display data for presenting the answer information, and causes a display device to display the display data.

### CROSS-REFERENCE TO RELATED APPLICATION

The present application claims priority based on Japanese Patent Application No. 2024-171204, filed September 30, 2024, the content of which is incorporated herein by reference.

### FIELD

The embodiments disclosed in this specification and the drawings relate to a medical information processing device, a medical information processing method, and a storage medium.

### BACKGROUND

Conventionally, in medical institutions such as hospitals, diseases suffered by patients are treated using SOAP (Subjective, Objective, Assessment, Plan). That is, doctors at hospitals perform evaluation (analysis and consideration) on the basis of subjective information on patient’s complaints, such as information obtained from face-to-face consultations or information written by the patient on a medical questionnaire and objective information such as numerical values of test results of the patient and examination results to make a definitive diagnosis to identify the disease suffered by the patient and develop a treatment plan to treat the disease.

However, when a doctor evaluates (analyzes, considers) the disease a patient suffers from, it is possible that the doctor may make an error in determination or that there may be some items that need to be reconfirmed with the patient in order to identify the disease (definitive diagnosis). This may be caused by, for example, the patient forgetting the main complaint that the patient should declare, the doctor forgetting items to confirm, or other reasons that necessary information is missing. Furthermore, although the patient declares to the doctor the details of their poor physical condition (information such as when symptoms started, what they felt like, and how frequently they occurred) as the main complaint, this main complaint is based on the patient’s subjective opinion (physical condition, mood, pain) and is also likely to be influenced by factors such as the lack of logical association with date, time, and behavior.

For this reason, with the conventional SOAP method, the quality of medical care provided to patients is affected by the quality of information collected by doctors from the patients. This is because not only the analysis, consideration (speculation) and identification of diseases by doctors, but also medical procedures by non-doctor medical professionals such as image diagnosticians, nurses, and rehabilitation specialists are performed on the basis of instructions from doctors and patient information provided by doctors. In addition, it takes time and effort for doctors to double-check with patients about missing information and for non-doctor medical professionals to check with doctors about necessary information.

In recent years, there has been consideration to create a personal large language model with patient information by adding patient’s personal information to a large language model (LLM) and use the same in the medical field. In addition, there has also been consideration to combine Retrieval Augmented Generation (RAG) technology, which improves the accuracy and reliability of a generated Artificial Intelligence (AI) model using information obtained from outside.

### BRIEF DESCRIPTION OF THE DRAWINGS

FIG. 1 is a diagram showing the configuration of a medical information processing system including a medical information processing device according to an embodiment, and an example of a usage environment of the medical information processing device.

FIG. 2 is a diagram showing an example of a functional configuration of a medical information processing device according to an embodiment.

FIG. 3 is a diagram showing an example of a display screen that presents questions to the medical information processing device according to an embodiment and answers thereto.

FIG. 4 is a flowchart showing an example of a flow of processing for updating a patient digital twin in the medical information processing device according to an embodiment.

FIG. 5 is a flowchart showing an example of a flow of processing at the time of answering a question in the medical information processing device according to an embodiment.

FIG. 6 is a diagram showing an example of questions to the medical information processing device according to an embodiment and answers thereto.

### DETAILED DESCRIPTION

A medical information processing device of an embodiment is a medical information processing device that receives a question for a patient from a medical professional and answers on behalf of the patient using a digital twin that reproduces the condition of the patient, the digital twin being an AI model trained by adding information on the patient to at least a large language model, the medical information processing device including processing circuitry. The processing circuitry receives the question, acquires at least medical condition information that represents the medical condition of the patient, updates the digital twin on the basis of the medical condition information, uses the digital twin to output answer information representing the answer to the received question, generates display data for presenting the answer information, and causes a display device to display the display data.

Hereinafter, a medical information processing device, a medical information processing method, and a storage medium according to an embodiment will be described with reference to the drawings.

The medical information processing device is a device that answers questions about a patient from, for example, a doctor or a medical professional other than a doctor (such as an image diagnostician, a nurse, or a rehabilitation specialist who performs medical procedures on a patient on the basis of instructions from the doctor and information on the patient provided by the doctor: hereinafter simply referred to as a “medical professional”) on behalf of the patient. In the following description, when there is no distinction between a doctor and a medical professional, they will be referred to as “medical professionals.” In the following description, a medical information processing system including the medical information processing device that answers questions from doctors and medical professionals will be described.

### [Usage environment of medical information processing system]

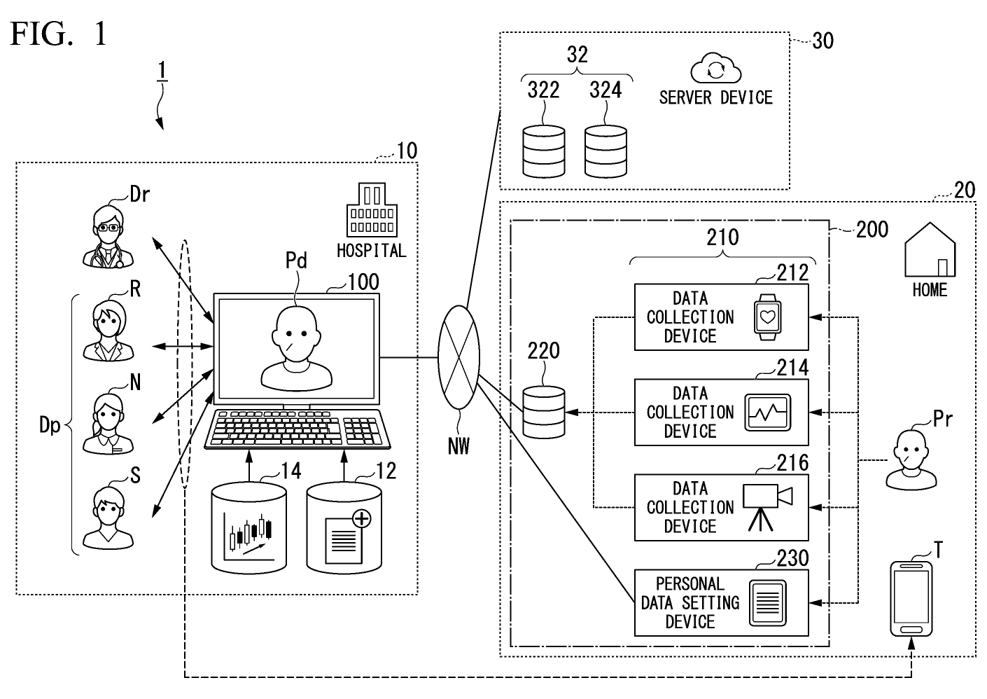

FIG. 1 is a diagram showing the configuration of a medical information processing system including a medical information processing device according to the embodiment, and an example of a usage environment of the medical information processing device. The medical information processing system 1 is configured to combine a medical information processing device 100 installed in a medical institution such as a hospital 10, a patient information collection device 200 installed in a home 20 of an actual patient (hereinafter referred to as a “real patient”) Pr who is a subject of diagnosis, and a server device 30 including a patient information storage device 32 that stores various types of information related to the real patient Pr. In the configuration of the medical information processing system 1 shown in FIG. 1, a case in which three data collection devices 210, a life log collection device 220, and a personal data setting device 230 are included is illustrated as an example of the patient information collection device 200.

Real patients Pr who are the subjects of diagnosis include, for example, patients who are suffering or have suffered from some disease, such as patients suffering from chronic diseases (chronic disease patients), patients suffering from lifestyle-related diseases, patients undergoing follow-up observation after undergoing some medical procedures, and patients undergoing observation to prepare for surgery, and a plurality of people whose health is managed by doctors or medical professionals, such as those who have been diagnosed with diseases during a health checkup and healthy people who wish to manage their own health.

In the medical information processing system 1, the medical information processing device 100, the patient information collection device 200, and the server device 30 communicate via a network NW. The network NW is a wireless communication network that includes, for example, the Internet, a wide area network (WAN), a local area network (LAN), a provider device, a wireless base station, and the like. In FIG. 1, although the patient information collection device 200 collects information on one real patient Pr, there may be a plurality of real patients Pr in the same home 20. Furthermore, although FIG. 1 shows one patient information collection device 200 connected to each of the medical information processing device 100 and the server device 30 via the network NW, a plurality of patient information collection devices 200, i.e., each patient information collection device 200 installed in the home 20 of each of a plurality of real patients Pr, may be connected to each of the medical information processing device 100 and the server device 30 via the network NW.

The patient information collection device 200 collects records of the living conditions of the real patient Pr (hereinafter referred to as “life logs”) measured and acquired by the data collection devices 210. The life logs include information and data such as the weight, heart rate, respiratory rate, blood pressure, dietary content, movement history, activity status, and activities performed under the guidance of a doctor or medical professional of the real patient Pr. The patient information collection device 200 sends the life logs collected by the data collection device 210 to the server device 30 (which may be the medical information processing device 100) via the network NW using the life log collection device 220. The patient information collection device 200 sends personal data set by the real patient Pr in the personal data setting device 230 to the server device 30 (which may be the medical information processing device 100) via the network NW.

The life logs are an example of “medical condition information,” “patient information,” “living records,” and “second patient information.” The personal data is an example of “medical condition information,” “patient information,” “second patient information,” and “third patient information.”

Each of the data collection devices 210 communicates with the life log collection device 220 and outputs (sends) an acquired life log of the real patient Pr to the life log collection device 220. Communication between the life log collection device 220 and the data collection device 210 may be, for example, wired communication using a communication cable, or short-range wireless communication such as Bluetooth (registered trademark). Each data collection device 210 outputs (sends) the acquired life log of the real patient Pr to the life log collection device 220 at predetermined time intervals.

FIG. 1 shows, as an example of the data collection devices 210, a wristwatch-type wearable device 212 owned by the real patient Pr, a vital measuring device 214 that measures vital data such as the body temperature, blood pressure, and electrocardiogram (heart rate) of the real patient Pr, and an imaging device 216 that captures images of the real patient Pr from a predetermined position in the home 20. The wearable device 212 is worn by the real patient Pr to measure the amount of physical activity such as walking and exercise in the life of the real patient Pr, and outputs (sends) the amount of activity to the life log collection device 220 as a life log. The vital measuring device 214 measures biological data including vital data such as the body temperature, blood pressure, and electrocardiogram (heart rate) of the real patient Pr, and outputs (sends) the measured results to the life log collection device 220 as a life log. The vital data may be measured by the wearable device 212 worn by the real patient Pr. The vital measuring device 214 may be, for example, a mobile terminal that combines the functions of a mobile phone using an existing mobile communication network with the functions of a personal digital assistant (PDA), so-called a smartphone or a tablet terminal. The imaging device 216 captures an image of the real patient Pr in the home 20, and outputs (sends) data of the captured image to the life log collection device 220 as a life log. The imaging device 216 may perform predetermined image processing on the captured image to analyze, for example, the body temperature of the real patient Pr, and output (sends) information representing the analysis result to the life log collection device 220 as a life log. The imaging device 216 may perform predetermined image recognition processing on the captured image to ascertain, for example, eating, drinking, and smoking states of the real patient Pr, and output (send) information representing the ascertained result to the life log collection device 220 as a life log. The data collection devices 210 shown in FIG. 1 are merely an example, and the data collection devices 210 include various devices that acquire life logs of the real patient Pr. For example, the data collection devices 210 may be a pedometer (registered trademark) carried by the real patient Pr, a blood pressure monitor used for the real patient Pr to voluntarily measure blood pressure, and the like. For example, the data collection devices 210 may be devices for checking whether the real patient Pr has any of various disorders, such as a salinity sensor capable of measurement of salinity concentration and various sensors capable of measuring urine, feces, saliva, blood, etc. The life log output by each data collection device 210 to the life log collection device 220 is associated with information on the time when the life log has been measured (acquired).

For example, the data collection devices 210 may store a life log measured while the real patient Pr is out in a memory (storage) (not shown), and when the real patient Pr returns to the home 20 and communication with the life log collection device 220 is established, output (send) the life log stored in the memory (not shown) to the life log collection device 220. The memory (not shown) may be realized, for example, by a semiconductor memory element such as a read only memory (ROM), a random access memory (RAM), or a flash memory.

The data collection device 210 is an example of a “collection device.”

The life log collection device 220 receives a life log output (sent) by each data collection device 210, and sends the received life log to the server device 30 (which may be the medical information processing device 100) via the network NW. The life log collection device 220 may include, for example, a storage device (storage) (not shown), collect life logs output (sent) by each data collection device 210, and send the collected life logs to the server device 30 (which may be the medical information processing device 100) via the network NW at predetermined time intervals. The storage device (not shown) is realized by, for example, a semiconductor memory element such as a ROM, a RAM, or a flash memory, a hard disk drive (HDD), or the like.

The life log collection device 220 is an example of a “collection device.”

Personal data is set in the personal data setting device 230 by the real patient Pr. The personal data setting device 230 is a terminal device (e.g., a tablet terminal) that exchanges personal data with the server device 30 (which may be the medical information processing device 100) via the network NW. The personal data is data (information) about the real patient Pr that a doctor Dr has not interviewed, such as personal information, past medical history, medications being taken, and information about allergies. Although FIG. 1 shows a configuration in which the personal data setting device 230 directly exchanges data and information (personal data) with the server device 30 (which may be the medical information processing device 100) via the network NW, this is merely an example, and the personal data setting device 230 may be configured to output (send) personal data set by the real patient Pr to the life log collection device 220 and send the personal data from the life log collection device 220 to the server device 30 (which may be the medical information processing device 100).

The personal data setting device 230 is an example of a “collection device.”

The server device 30 is, for example, a server device on the network NW incorporated in a network or a cloud computing system of a medical institution. The server device 30 stores the life logs and personal data sent by the patient information collection device 200 and transmitted over the network NW in the patient information storage device 32. More specifically, the server device 30 stores the history of the life logs sent by the life log collection device 220 in a life log storage device 322, and stores the history of the personal data sent by the personal data setting device 230 in a personal data storage device 324. The patient information storage device 32, that is, the life log storage device 322 and the personal data storage device 324, are realized by, for example, semiconductor memory elements such as a ROM, a RAM, and a flash memory, and a hard disk drive (HDD), and the like. In response to a request from the medical information processing device 100, the server device 30 sends life logs of the real patient Pr stored in the life log storage device 322 and personal data of the real patient Pr stored in the personal data storage device 324 to the medical information processing device 100 via the network NW. Although FIG. 1 shows a case in which one server device 30 is connected to the network NW, this is merely an example, and the server device 30 may include a plurality of server devices connected to the network NW, or may include other server devices connected to other networks to which the server device 30, the medical information processing device 100, and the patient information collection device 200 can be connected.

Although FIG. 1 shows that the server device 30 is configured to store life logs and personal data sent by the patient information collection device 200 in the patient information storage device 32 (life log storage device 322 or personal data storage device 324), the server device 30 may be configured to realize some or all of the functions of the medical information processing device 100 which will be described below. In this case, the main device of the medical information processing device 100 and the server device 30 in which some of the functions have been realized communicate with each other via the network NW. Here, if the server device 30 is configured to realize all of the functions of the medical information processing device 100, it is sufficient that at least a display device and an input interface are installed in the hospital 10, and the server device 30, which is the main device of the medical information processing device 100, is configured to communicate with the display device and the input interface via the network NW.

The server device 30 and the life log storage device 322 and personal data storage device 324 included in the patient information storage device 32 provided in the server device 30 may be an example of a “collection devices.”

The medical information processing device 100 answers questions from doctors (including medical professionals) to the real patient Pr on behalf of the real patient Pr. The medical information processing device 100 is realized by a computer device such as a personal computer (PC) installed in an examination room of the hospital 10, for example. FIG. 1 shows an example in which the medical information processing device 100 is realized by a personal computer located in the hospital 10. When the medical information processing device 100 is realized by a personal computer, an input interface for the doctor Dr to input operations and information (questions) of the medical information processing device 100, a display device for presenting information (answers) to the doctor Dr, and the like are connected to the medical information processing device 100. The input interface and the display device may be connected to the medical information processing device 100 through wireless communication.

The input interface is realized by, for example, a mouse, a keyboard, a touch panel, a microphone, or the like. When the input interface is a touch panel, the input interface may be formed integrally with the display device connected to the medical information processing device 100. In this specification, the input interface is not limited to an interface having physical operation parts such as the mouse and keyboard described above. For example, examples of the input interface include an electrical signal processing circuitry that receives an electrical signal corresponding to an input operation from an external input device provided separately from the medical information processing device 100 and outputs the electrical signal to the medical information processing device 100. The display device is, for example, a liquid crystal display (LCD), a cathode ray tube (CRT) display, an organic electroluminescence (EL) display, or the like.

The medical information processing device 100 may be realized by a computer device such as a terminal device (e.g., a tablet terminal) carried by a medical professional Dp belonging to the hospital 10. In this case, the medical professional Dp operates the medical information processing device 100 and inputs information (questions) using an input interface built into the terminal device, and information (answer) is presented to the medical professional Dp by a display device (e.g., a liquid crystal display (LCD)) built into the terminal device. When the medical information processing device 100 is realized by a terminal device, the input interface may be a microphone built into the terminal device, and information (answer) may be presented to the medical professional Dp by a speaker built into the terminal device instead of or in addition to the display device.

The medical information processing device 100 may be realized by a server device (which may be the server device 30) built into a network NW, a network (not shown), or a cloud computing system. In this case, at least an input interface and a display device may be installed in an examination room, and the server device, which is the main device of the medical information processing device 100, communicates with the input interface and the display device via the network NW, the network (not shown), or the like. Furthermore, only some functions of the medical information processing device 100, which will be described later, may be realized by a server device, and in this case, the server device, which is the main device of the medical information processing device 100, and the server device in which some functions are realized communicate with each other via the network NW, the network (not shown), or the like. The network (not shown), like the network NW, may include the Internet, a WAN, a LAN, a provider device, a wireless base station, etc., for example.

The medical information processing device 100 is not limited to being realized as an independent device such as a computer device or a terminal device, and may be realized, for example, as a function of a medical system composed of different devices and equipment. As a medical system, for example, an electronic medical record system that manages the results of diagnoses and examinations of patients, previously performed, in the form of a medical record (electronic medical record), or a database system such as a picture archiving and communication system (PACS) that manages data of medical images of patients is conceivable. Furthermore, the medical information processing device 100 and the functions thereof may be realized as a medical information integration system that integrates information managed by each medical system and presents the same to doctors, or as a function of a measurement application (medical information integration application) for realizing that function. In such cases, at least the input interface and the display device are shared with devices and equipment such as the medical system that realizes the functions of the medical information processing device 100.

The medical information processing device 100 answers questions for the real patient Pr, for example, using an artificial intelligence (AI) model of the real patient Pr that has been trained by adding the personal information of the real patient Pr to a large language model (LLM). The AI model of the real patient Pr is realized, for example, as a digital twin (Patient Twin: hereinafter referred to as “Patient Digital Twin”) that reproduces the state of the real patient Pr as a twin in a digital space. The AI model of the real patient Pr may be realized as a persona that reproduces the characteristics of the real patient Pr. In the following description, it is assumed that the medical information processing device 100 answers questions for the real patient Pr on the basis of the state of the patient digital twin. The patient digital twin may be generated in the medical information processing device 100 or in the server device 30.

FIG. 1 shows an example of a case in which a patient digital twin is generated on the basis of actual information and data related to the disease suffered by the real patient Pr, including, for example, medical record data stored in an electronic medical record system 12 that manages medical records (electronic medical records) related to the main complaints of the real patient Pr and the medical condition of the real patient Pr examined by a doctor, and examination data stored in an examination management system 14 that manages the examination results of the real patient Pr. The patient digital twin may be generated on the basis of medical image data stored in a picture archiving and communication system (PACS) that manages medical images of the real patient Pr. FIG. 1 also shows an example of a situation in which, for example, the doctor Dr who is the attending physician of the real patient Pr, and medical professionals Dp other than the doctor Dr, such as a radiologist R, a nurse N, and a rehabilitation specialist S, ask questions to the medical information processing device 100, and a digital patient Pd, which is a schematic representation of the patient digital twin in the medical information processing device 100, answers each of the questions. In other words, the figure shows an example in which the hospital 10 is in a state equivalent to having the digital patient Pd present at all times, and the doctor Dr and medical professionals Dp (radiologist R, nurse N, rehabilitation specialist S, etc.) can ask the real patient Pr (actually, the digital patient Pd) questions about, for example, the amount of the main complaints that the patient has forgotten or confirmation items that the doctor Dr was unable to confirm initially, at any time, and receive answers.

When the medical record data stored in the electronic medical record system 12 or the examination data stored in the examination management system 14 is updated, the medical information processing device 100 acquires the updated medical record data and examination data, and updates the patient digital twin on the basis of the acquired medical record data and examination data. Furthermore, when life logs collected by the data collection device 210 or personal data set in the personal data setting device 230 is updated, the medical information processing device 100 acquires the updated life logs and personal data from the server device 30 via the network NW, and updates the patient digital twin on the basis of the acquired life logs and personal data. Here, the updated patient digital twin is a digital twin that includes (reproduces) not only information and data about the disease suffered by the real patient Pr who is the subject of diagnosis this time, but also the living conditions of the real patient Pr. Accordingly, the medical information processing device 100 can answer subsequent questions about the real patient Pr on the basis of the state of the updated patient digital twin (a digital twin that also reproduces the living conditions of the real patient Pr). Moreover, there are concerns that the main complaints of real patients Pr may not be related logically to dates, times, actions, etc., but the updated patient digital twin here is updated on the basis of at least the life log that is collection of records of the living conditions of the real patient Pr, and is therefore a digital twin that does not contain temporal inconsistencies or inaccurate information. In other words, the updated patient digital twin provides more accurate answers because it eliminates the possibility of hallucinations of the AI model in the large language model (LLM), for example.

When the medical information processing device 100 cannot answer a question content posed to the real patient Pr by the doctor Dr or medical professional Dp using the current patient digital twin, the medical information processing device 100 acquires the current (i.e., the latest) medical record data, examination data, life logs collected by the data collection device 210 up to the present time, and personal data set in the personal data setting device 230, and updates the patient digital twin on the basis of the acquired medical record data, examination data, life logs, and personal data. Then, the medical information processing device 100 answers the question posed by the doctor Dr or medical professional Dp on the basis of the updated state of the patient digital twin. At this time, the medical information processing device 100 adds (stores) information relating to the question content posed to the real patient Pr by the doctor Dr or medical professional Dp and the answer in, for example, the electronic medical record system 12. In this case, the medical information processing device 100 distinguishes between the medical record data already stored in the electronic medical record system 12 and the information associated with the question content and answer to be added (hereinafter referred to as “QA information”) and adds (stores) the same in the electronic medical record system 12.

For example, when the doctor Dr or medical professional Dp issues necessary guidance for the real patient Pr to cure a disease, the medical information processing device 100 notifies the real patient Pr of the amount of this guidance. The amount of guidance may include, for example, information regarding the progress of a surgery performed on the real patient Pr and follow-up after discharge. FIG. 1 schematically shows an example of a state in which the amount of guidance issued by the doctor Dr or the medical professional Dp is transmitted to a terminal device T (for example, a mobile terminal) owned by the real patient Pr. The medical information processing device 100 may notify the real patient Pr of the amount of guidance issued by the doctor Dr or the medical professional Dp by transmitting the same to the personal data setting device 230. At this time, the medical information processing device 100 adds (stores) information representing the amount of guidance issued by the doctor Dr or the medical professional Dp for the real patient Pr, for example, in the electronic medical record system 12. In this case, the medical information processing device 100 distinguishes between the medical record data (which may include QA information) already stored in the electronic medical record system 12 and the information representing the amount of guidance to be added (hereinafter referred to as “instruction information”) and adds (stores) the same in the electronic medical record system 12.

[Functional configuration of medical information processing device]

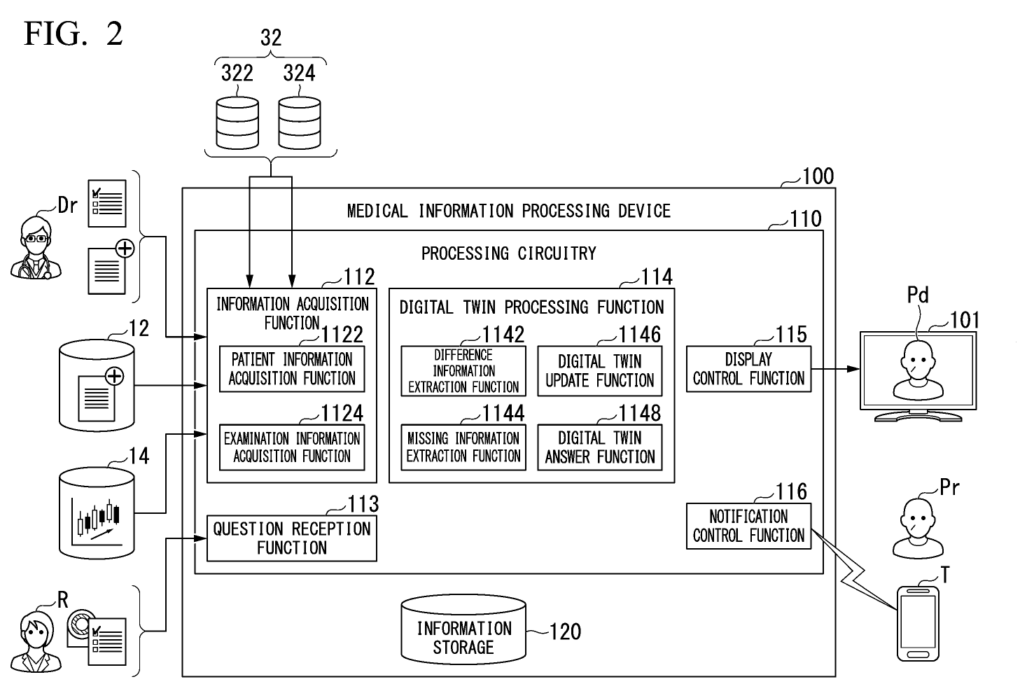

FIG. 2 is a diagram showing an example of the functional configuration of the medical information processing device 100 according to an embodiment. FIG. 2 also shows components in the hospital 10, the home 20, and the server device 30 related to the function of the medical information processing device 100 to answer questions for the real patient Pr using a patient digital twin.

The medical information processing device 100 includes, for example, processing circuitry 110 and an information storage 120.

The information storage 120 stores various types of information and data when the medical information processing device 100 answers questions from the doctor Dr or a medical professional Dp for the real patient Pr. The information storage 120 stores the latest patient digital twin for the real patient Pr, and medical record data, examination data, life logs, personal data, and the like corresponding to this patient digital twin. The information storage 120 is realized, for example, by semiconductor memory elements such as a ROM, a RAM, and a flash memory, a hard disk drive (HDD), and the like.

The processing circuitry 110 executes, for example, processing such as an information acquisition function 112, a question reception function 113, a digital twin processing function 114, a display control function 115, and a notification control function 116. The information acquisition function 112 executes processing such as a patient information acquisition function 1122 and an examination information acquisition function 1124. The digital twin processing function 114 executes processing such as a difference information extraction function 1142, a missing information extraction function 1144, a digital twin update function 1146, and a digital twin answer function 1148.

The functional configuration of the processing circuitry 110 shown in FIG. 2 is a functional configuration in the case in which the latest patient digital twin of the real patient Pr is stored in the information storage 120, that is, a patient digital twin that reproduces the state of the real patient Pr has already been generated. When a patient digital twin of the real patient Pr has not been generated, it will be newly generated in the medical information processing device 100, the server device 30, or another information processing device. When a patient digital twin of the real patient Pr is newly generated in the medical information processing device 100, for example, a patient digital twin generation function will be added to the functional configuration of the processing circuitry 110 shown in FIG. 2. However, in this case, the generation of the patient digital twin in the patient digital twin generation function can be performed using existing technology of adding the personal information of the real patient Pr to a large language model (LLM) to generate an AI model (retrieval augmented generation (RAG) technology may be combined), for example. Therefore, detailed descriptions of the functional configuration that realizes the function of generating a patient digital twin in the medical information processing device 100 or the processing circuitry 110, and operation and processing thereof will be omitted.

The processing circuitry 110 realizes the functions of the information acquisition function 112 (including the patient information acquisition function 1122 and the examination information acquisition function 1124), the question reception function 113, the digital twin processing function 114 (including the difference information extraction function 1142, the missing information extraction function 1144, the digital twin update function 1146, and the digital twin answer function 1148), the display control function 115, and the notification control function 116 by a hardware processor executing a program (software) stored in a memory (storage) (not shown), for example. The memory (not shown) is realized by, for example, a semiconductor memory element such as a ROM, a RAM, or a flash memory, a hard disk drive (HDD), or an optical disc.

The hardware processor refers to circuitry such as a central processing unit (CPU), a graphics processing unit (GPU), large scale integration (LSI), a system on chip (SOC), an application specific integrated circuit (ASIC), a programmable logic device (e.g., a simple programmable logic device (SPLD) or a complex programmable logic device (CPLD), a field programmable gate array (FPGA)). Instead of storing the program in the memory (not shown), the program may be directly built into the circuit of the hardware processor. In this case, the hardware processor realizes each function by reading and executing the program built into the circuit. The hardware processor is not limited to being configured as a single circuit, but may be configured as a single hardware processor by combining a plurality of independent circuits to realize each function. A plurality of components may be integrated into a single hardware processor to realize each function. A plurality of components may be incorporated into one dedicated LSI to realize each function. Here, the program (software) may be stored in advance in a storage device (storage device having a non-transient storage medium) constituting a storage device such as a semiconductor memory element such as a ROM, a RAM, or a flash memory, or a hard disk drive (HDD), or may be stored in a removable storage medium (non-transient storage medium) such as a DVD or a CD-ROM and installed in a storage device (not shown) included in the medical information processing device 100 by setting the storage medium in a drive device included in the medical information processing device 100. The program (software) may be downloaded in advance from another computer device via a network (not shown) and installed in the storage device included in the medical information processing device 100. The program (software) installed in the storage device included in the medical information processing device 100 may be transferred to the processing circuitry 110 included in the medical information processing device 100 and executed.

The information acquisition function 112 acquires information and data (medical record data, life logs, examination data, and personal data) of the real patient Pr, and outputs the acquired information and data to the digital twin processing function 114.

The patient information acquisition function 1122 acquires information and data (which is also medical record data) regarding the main complaints of the real patient Pr input by the doctor Dr and the medical condition examined by the doctor Dr, and medical record data stored in the electronic medical record system 12. The main complaints of the real patient Pr include, for example, information and data representing the details of the poor physical condition that caused the real patient Pr to come to the clinic for treatment (information and data such as when symptoms began, what they felt like, and how frequently they occurred). Information and data regarding the medical condition examined by the doctor Dr include, for example, information and data representing the state of the real patient Pr observed by the doctor Dr, and information and data regarding the results of examination by the doctor Dr. FIG. 2 schematically shows, for example, a state in which medical record data is acquired from each of the doctor Dr and the electronic medical record system 12. The patient information acquisition function 1122 acquires a life log and personal data from the patient information storage device 32 (more specifically, the life log storage device 322 and the personal data storage device 324). FIG. 2 schematically shows, for example, a state in which a life log and personal data are acquired from each of the life log storage device 322 and the personal data storage device 324 included in the patient information storage device 32. The patient information acquisition function 1122 outputs the acquired medical record data, life log, and personal data (hereinafter, when the medical record data, the life log, and the personal data are not distinguished, they are referred to as “patient information”) to the digital twin processing function 114. The patient information acquisition function 1122 may store the acquired patient information in the information storage 120 and notify the digital twin processing function 114 of the patient information such that the digital twin processing function 114 acquires the patient information from the information storage 120.

The examination information acquisition function 1124 acquires examination information related to examinations performed on the real patient Pr. The examination information includes, for example, information on the treatment performed on the real patient Pr by the doctor Dr in an examination room and examination data stored in the examination management system 14 (for example, the numerical values of each examination item performed on the real patient Pr). The examination information may include, for example, medical image data of the real patient Pr captured by a medical image diagnostic device, such as a CT image captured by a computed tomography (CT) device, an MR image captured by a magnetic resonance imaging (MRI) device, or an ultrasound image (echo image) captured by an ultrasound diagnostic device, stored in a picture archiving and communication system (PACS), and information indicating findings diagnosed by the doctor Dr or an image diagnostician on the basis of the medical image. FIG. 2 schematically shows, for example, a state in which examination data is acquired from the examination management system 14. The examination information acquisition function 1124 outputs the acquired examination information to the digital twin processing function 114. The examination information acquisition function 1124 may store the acquired examination information in the information storage 120 and notify the digital twin processing function 114 of the examination information such that the digital twin processing function 114 acquires the examination information from the information storage 120.

The information acquisition function 112, the patient information acquisition function 1122, and the examination information acquisition function 1124 are an example of an “information acquirer.” Medical record data is an example of “symptom information,” “patient information,” and “first patient information.” Examination data is an example of “symptom information,” “patient information,” and “examination information.”

The information acquisition function 112 may output the patient information acquired by the patient information acquisition function 1122 and the examination information acquired by the examination information acquisition function 1124 to the display control function 115. The information acquisition function 112 may store the patient information and the examination information in the information storage 120 and notify the display control function 115 of the patient information and the examination information such that the display control function 115 acquires the patient information and the examination information from the information storage 120. In this case, when the medical information processing device 100 answers a question from the doctor Dr or the medical professional Dp, the display control function 115 can present the patient information and the examination information to the questioner.

The question reception function 113 receives a question from the doctor Dr or medical professional Dp with respect to the real patient Pr. FIG. 2 schematically shows a state in which a question with respect to the real patient Pr is received from, for example, a radiologist R. Any method may be used for receiving a question in the question reception function 113. For example, the doctor Dr or medical professional Dp may operate an input interface to input a question content to the medical information processing device 100. For example, if the voice uttered by the doctor Dr or medical professional Dp can be picked up by a microphone installed in an examination room and connected to the medical information processing device 100, or a microphone built into a terminal device, the question reception function 113 may receive voice information uttered by the doctor Dr or medical professional Dp into the microphone as information on the question content. In this case, the question reception function 113 receives the question content based on the voice uttered by the doctor Dr or medical professional Dp, for example, by using a large language model (LLM) provided in the patient digital twin or a natural language processing function in another large language model (LLM: not shown). The question reception function 113 outputs information or data representing the received question content (hereinafter referred to as “question information”) to each of the digital twin processing function 114, the information acquisition function 112, and the display control function 115. The question reception function 113 stores the received question information in the information storage 120, and notifies each of the digital twin processing function 114, the information acquisition function 112, and the display control function 115 of the question information such that each of the digital twin processing function 114, the information acquisition function 112, and the display control function 115 may acquire the question information from the information storage 120. In the medical information processing device 100, the information acquisition function 112 adds (stores) the question information in the electronic medical record system 12 as the question content in QA information.

As described above, the medical information processing device 100 notifies the real patient Pr of guidance from the doctor Dr or the medical professional Dp, for example. The guidance may be received by the question reception function 113. That is, the question reception function 113 may receive guidance for the real patient Pr in addition to questions from the doctor Dr or the medical professional Dp to the real patient Pr. In this case, the question reception function 113 may determine whether a voice uttered by the doctor Dr or the medical professional Dp is a question or guidance for the real patient Pr by a natural language processing function in the large language model (LLM) described above, for example. When the question reception function 113 receives guidance for the real patient Pr, the question reception function 113 outputs information or data representing the amount of guidance (guidance information) to each of the notification control function 116 and the information acquisition function 112. The question reception function 113 may store the received guidance information in the information storage 120 and notify the notification control function 116 and the information acquisition function 112 of the same such that the notification control function 116 and the information acquisition function 112 acquire the guidance information from the information storage 120. As a result, in the medical information processing device 100, the notification control function 116 notifies the real patient Pr of the guidance information, and the information acquisition function 112 adds (stores) the guidance information in the electronic medical record system 12.

The question reception function 113 is an example of a “receptor.”

The digital twin processing function 114 performs processing to answer questions for the real patient Pr using the patient digital twin. When the digital twin processing function 114 can answer a question content for the real patient Pr represented by question information output by the question reception function 113, for example, using the patient digital twin currently stored in the information storage 120, the digital twin processing function 114 answers the question using the current patient digital twin. When the digital twin processing function 114 cannot answer the question for the real patient Pr using the current patient digital twin, the digital twin processing function 114 updates the current patient digital twin on the basis of patient information and examination information output by the information acquisition function 112, and answers the question using the updated patient digital twin. More specifically, when the digital twin processing function 114 cannot answer the question for the real patient Pr using the current patient digital twin, the digital twin processing function 114 first updates the current patient digital twin on the basis of medical record data and examination data acquired by the information acquisition function 112, and answers the question using the updated patient digital twin. If the digital twin processing function 114 cannot answer the question for the real patient Pr even by using the updated patient digital twin, the digital twin processing function 114 further updates the updated patient digital twin on the basis of life logs and personal data acquired by the information acquisition function 112, and answers the question using the further updated patient digital twin. The digital twin processing function 114 outputs information or data (hereinafter referred to as “answer information”) representing the answer to the question for the real patient Pr to the display control function 115 such that the answer is presented to the questioner. In this manner, the medical information processing device 100 presents answers to questions about main complaints of the real patient Pr or the condition (such as a medical condition) of the real patient Pr that cannot be confirmed by the doctor Dr, thereby supporting the doctor Dr and the medical professional Dp in making diagnoses and medical procedures for the real patient Pr.

The difference information extraction function 1142 extracts differences between information and data such as medical record data, examination data, life logs, and personal data acquired by the information acquisition function 112 and corresponding information and data stored in the information storage 120. The difference information extraction function 1142 adds (stores) information on the extracted differences (difference information) in the information acquisition function 112 and notifies the digital twin update function 1146 of the same.

The difference information extraction function 1142 is an example of a “difference extractor.” Information and data such as medical record data, examination data, life logs, and personal data acquired by the information acquisition function 112 are an example of “new medical condition information.” Information and data such as medical record data, examination data, life logs, and personal data stored in the information storage 120 is an example of “previous medical condition information.”

The missing information extraction function 1144 determines whether it is possible to answer the question content for the real patient Pr represented by the question information output by the question reception function 113 using the current patient digital twin. Then, if the missing information extraction function 1144 determines that the question can be answered, the missing information extraction function 1144 outputs the current question content to the digital twin answer function 1148. On the other hand, if the missing information extraction function determines that the question cannot be answered, the missing information extraction function 1144 extracts information or data (missing information) that is missing to answer the current question content. More specifically, the missing information extraction function 1144 extracts the missing information by comparing the information or data corresponding to the current question content with information or data such as medical record data, examination data, life logs, and personal data stored in the information storage 120. Then, the missing information extraction function 1144 instructs the information acquisition function 112 to acquire information or data such as the latest medical record data, examination data, life log, and personal data. As a result, the information acquisition function 112 (more specifically, the patient information acquisition function 1122 and the examination information acquisition function 1124) acquires the latest patient information and examination information. The missing information extraction function 1144 notifies the digital twin update function 1146 that there is information or data (missing information) that is missing to answer the current question content.

The missing information extraction function 1144 is an example of a “missing information extractor.”

When the digital twin update function 1146 is notified by the difference information extraction function 1142 that difference information has been added (stored) in the information acquisition function 112, or notified by the information acquisition function 112 that the latest patient information or examination information has been stored in the information acquisition function 112, the digital twin update function 1146 updates the patient digital twin stored in the information acquisition function 112 on the basis of the medical record data, examination data, life logs, and personal data stored in the information acquisition function 112. That is, the digital twin update function 1146 updates the patient digital twin stored in the information acquisition function 112 to a patient digital twin that includes newer medical record data, examination data, life logs, and personal data. The digital twin update function 1146 stores the updated patient digital twin in the information acquisition function 112. The digital twin update function 1146 notifies the digital twin answer function 1148 that the patient digital twin has been updated.

The digital twin update function 1146 is an example of an “updater.”

The digital twin answer function 1148 uses the patient digital twin stored in the information acquisition function 112 to answer the question content for the real patient Pr, which is represented by the question information output by the question reception function 113. The digital twin answer function 1148 outputs information or data (answer information) representing the answer to the question to the display control function 115. Furthermore, the digital twin answer function 1148 outputs the answer information to the information acquisition function 112. The digital twin answer function 1148 may store the answer information in the information storage 120 and notify the information acquisition function 112 of the same such that the information acquisition function 112 acquires the answer information from the information storage 120. In the medical information processing device 100, the answer information is associated with the corresponding question content as an answer in QA information by the information acquisition function 112, and is added (stored) in the electronic medical record system 12.

The digital twin answer function 1148 is an example of an “answer unit.”

The display control function 115 generates a display image for presenting the information or data (question information) representing the question content received by the question reception function 113 and the information or data (answer information) representing the answer to the question output by the digital twin processing function 114 (more specifically, the digital twin answer function 1148) to the doctor Dr or medical professional Dp who asked the question of the real patient Pr. The display control function 115 presents the question content and the answer thereto to the doctor Dr or medical professional Dp by displaying the generated display image on a display device. FIG. 2 schematically shows, for example, a state in which a question is being answered by displaying a display image of a digital patient Pd on a display device 101 (e.g., a liquid crystal display (LCD)) connected to the medical information processing device 100.

The method of presenting a question content and an answer thereto to the doctor Dr or medical professional Dp in the display control function 115 is not limited to the method of displaying the display image of the digital patient Pd shown in FIG. 2. For example, the display control function 115 may be in a format in which a chatbot automatically answers questions.

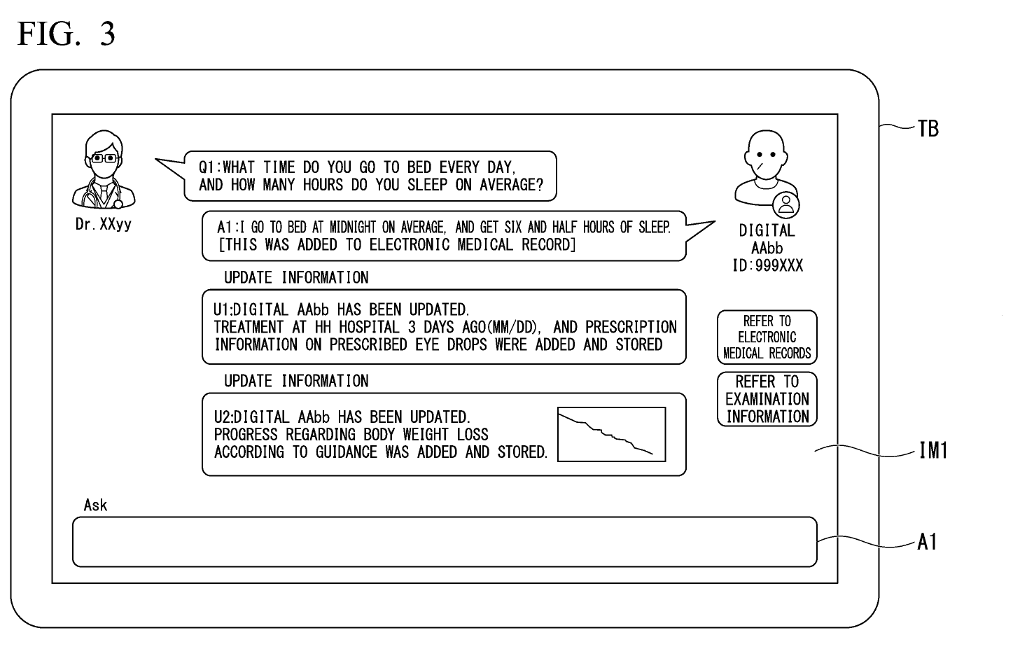

Here, an example of display by the display control function 115 in this case will be described. FIG. 3 is a diagram showing an example of a display screen presenting a question to the medical information processing device 100 and an answer thereto according to the embodiment. FIG. 3 shows an example of a display screen IM1 when a display image generated by the display control function 115 is displayed on a display device such as a liquid crystal display (LCD) incorporated in a tablet terminal TB carried by a doctor Dr (=XXyy). More specifically, the display screen IM1 shown in FIG. 3 shows an example of a case in which, when the doctor Dr inputs a question in a natural language, “What time do you go to bed every day, and how many hours do you sleep on average?,” in a question input area A1, the digital patient Pd (=AAbb) that is a schematic representation of a patient digital twin answers in a natural language, “I go to bed at midnight on average, and get six and a half hours of sleep.” Furthermore, the display screen IM1 shown in FIG. 3 shows that the question content and the answer this time are answers from the patient digital twin that have been acquired and updated by acquiring a life log, that is, information that has not been stored as medical record data in the electronic medical record system 12 so far, and therefore this QA information has been added to and stored in the electronic medical record system 12. Furthermore, the display screen IM1 shown in FIG. 3 shows an example of a case in which update information is displayed to notify that information on “medication prescribed at another hospital” and data on “body weight change according to weight loss guidance” obtained from the life log acquired when updating the patient digital twin have been added to and stored in the electronic medical record system 12. More specifically, the display screen IM1 shown in FIG. 3 shows update information indicating that information “3 days ago (MM/DD) the patient visited HH hospital and received treatment, and eye drops were prescribed” was added to and stored in the electronic medical record system 12, and data on “body weight change according to weight loss guidance” was added to and stored in the electronic medical record system 12 as “a graph of change in body weight as the real patient Pr tried to lose weight according to guidance.” Furthermore, the display screen IM1 shown in FIG. 3 shows an example of a button operated (for example, tapped or long pressed) by the doctor Dr when checking information and data (including updated information and data) of the electronic medical record system 12 and the examination management system 14. This allows the doctor Dr (which may be a medical professional Dp) to obtain information and data regarding the content (question content) that the doctor wants to check when making a diagnosis even when the real patient Pr has not visited the hospital, that is, when the doctor Dr cannot directly ask questions.

As described above, the medical information processing device 100 may, for example, receive a question posed to the real patient Pr by voice from the doctor Dr or medical professional Dp. In this case, the display control function 115 generates, for example, voice data (which is also answer information) for answering the question by voice, instead of or in addition to a display image described above. The display control function 115 then presents the answer to the question to the doctor Dr or medical professional Dp by vocalizing the generated voice data through a speaker.

The display control function 115 is an example of a “display controller.” The display image is an example of “display data.”

Referring back to FIG. 1, the notification control function 116 notifies the real patient Pr of, for example, information or data (guidance information) representing the amount of guidance received by the question reception function 113. The notification control function 116 notifies the real patient Pr of the guidance information, for example, by sending an e-mail or the like to the terminal device T of the real patient Pr. FIG. 2 schematically shows a state in which the real patient Pr is notified of guidance information, for example, by sending an e-mail or the like to the terminal device T of the real patient Pr.

The method of notifying of guidance information in the notification control function 116 is not limited to the method of sending the guidance information to the terminal device T shown in FIG. 2. For example, the notification control function 116 may generate a display image for presenting the amount of guidance represented by the guidance information to the real patient Pr, and transmit the generated display image to the personal data setting device 230 via the network NW. In this case, the display image transmitted via the network NW is displayed on a display device (e.g., a liquid crystal display (LCD)) built into the personal data setting device 230, and the amount of notifications are presented (notified) to the real patient Pr.

[Processing of medical information processing device]

Next, an example of a flow of processing in the medical information processing device 100 will be described.

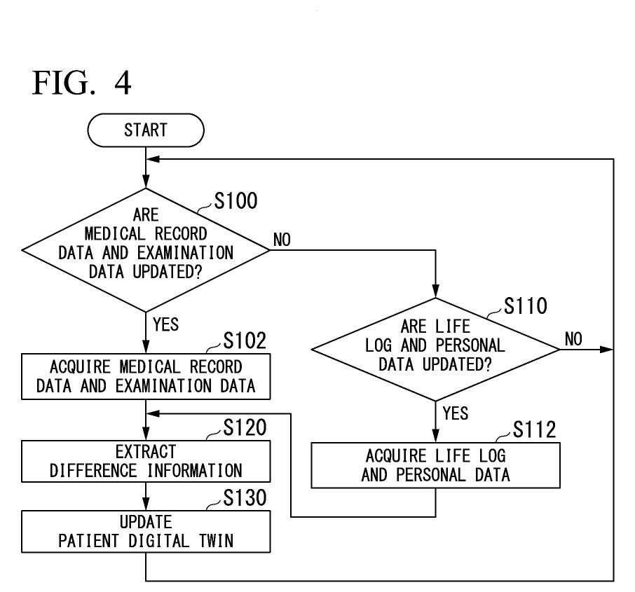

As described above, when medical record data stored in the electronic medical record system 12, examination data stored in the examination management system 14, a life log collected by the data collection device 210, or personal data set in the personal data setting device 230 has been updated, the medical information processing device 100 updates the patient digital twin on the basis of the updated information and data. First, an example of a flow of processing of the medical information processing device 100 when updating the patient digital twin on the basis of the updated information and data will be described. FIG. 4 is a flowchart showing an example of a flow of processing of updating the patient digital twin in the medical information processing device 100 according to an embodiment.

The processing circuitry 110 included in the medical information processing device 100 checks whether the medical record data or examination data has been updated (step S100). When it is confirmed that either or both of the medical record data and the examination data have been updated in step S100, the processing circuitry 110 acquires either or both of the updated medical record data and the examination data through the information acquisition function 112 (step S102). More specifically, the patient information acquisition function 1122 in the processing circuitry 110 acquires the updated medical record data, and the examination information acquisition function 1124 in the processing circuitry 110 acquires the updated examination data. Then, the information acquisition function 112 outputs either or both of the acquired medical record data and the examination data to the digital twin processing function 114, and the processing circuitry 110 proceeds to step S120.

On the other hand, if it is confirmed in step S100 that the medical record data and the examination data have not been updated, the processing circuitry 110 checks whether the life log and the personal data have been updated (step S110). If it is confirmed in step S110 that neither the life log nor the personal data have been updated, the processing circuitry 110 returns the processing to step S100 and repeats the processing of step S100 (which may include the processing of step S110) to continue checking whether any one or more pieces of information or data among the medical record data, the examination data, the life log, and the personal data have been updated.

On the other hand, if it is confirmed in step S110 that the life log and the personal data have been updated, the processing circuitry 110 acquires either or both of the updated life log and the personal data through the information acquisition function 112 (step S112). More specifically, the patient information acquisition function 1122 acquires either or both of the updated life log and personal data from the server device 30 (the life log storage device 322 and the personal data storage device 324 included in the patient information storage device 32) via the network NW. Then, the information acquisition function 112 outputs either or both of the acquired life log and personal data to the digital twin processing function 114, and the processing circuitry 110 advances the processing to step S120.

If it is confirmed in the processing of step S100 or step S110 that one or more pieces of the information or data of the medical record data, examination data, life log, and personal data have been updated, the processing circuitry 110 extracts differences (difference information) between the acquired information or data and the corresponding information or data stored in the information storage 120 through the digital twin processing function 114 (more specifically, the difference information extraction function 1142) (step S120). The difference information extraction function 1142 adds (stores) the difference information in the information acquisition function 112 and notifies the digital twin update function 1146 of the same.

When the difference information extraction function 1142 notifies the information acquisition function 112 that the difference information has been added (stored), the processing circuitry 110 updates the patient digital twin stored in the information acquisition function 112 on the basis of one or more pieces of the updated medical record data, examination data, life log, and personal data stored in the information acquisition function 112 through the digital twin processing function 114 (more specifically, the digital twin update function 1146) (step S130). The digital twin update function 1146 stores the updated patient digital twin in the information acquisition function 112 and notifies the digital twin answer function 1148 of the same. The processing circuitry 110 then ends the processing of this flowchart in which the patient digital twin is updated on the basis of the updated information and data.

In this way, the processing circuitry 110 in the medical information processing device 100 updates the patient digital twin on the basis of the updated information and data. As a result, in the medical information processing device 100, the digital twin processing function 114 (more specifically, the digital twin update function 1146) in the processing circuitry 110 can answer questions about the real patient Pr received by the question reception function 113 using the newer state of the patient digital twin stored in the information acquisition function 112.

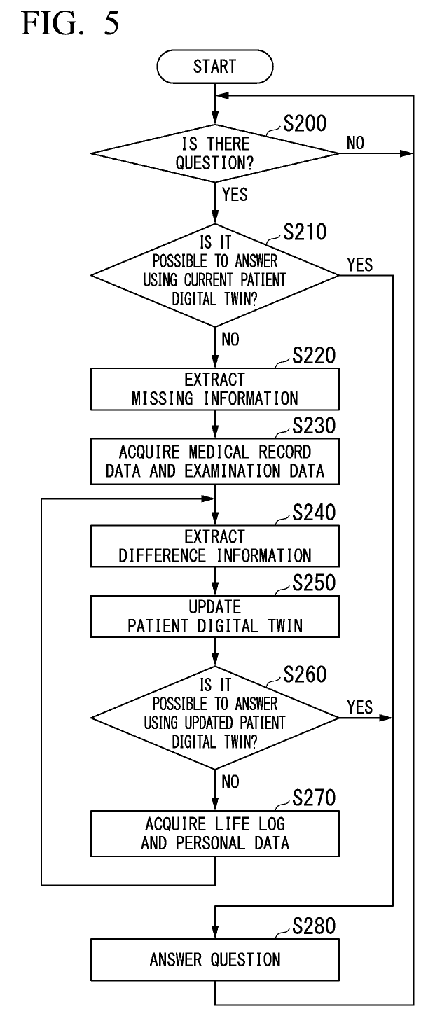

Next, an example of a flow of processing of the medical information processing device 100 when a doctor Dr or medical professional Dp asks a question about the real patient Pr will be described. FIG. 5 is a flowchart showing an example of a flow of processing when answering a question in the medical information processing device 100 according to an embodiment.

The processing circuitry 110 included in the medical information processing device 100 checks whether a question has been received in the question reception function 113 (step S200). If it is confirmed in step S200 that a question has not been received, the processing circuitry 110 repeats the checking processing in step S200.

On the other hand, if it is confirmed in step S200 that a question has been received, the question reception function 113 outputs question information representing the received question content to at least the digital twin processing function 114. Then, the processing circuitry 110 uses the current patient digital twin stored in the information storage 120 through the digital twin processing function 114 (more specifically, the missing information extraction function 1144) to determine whether it is possible to answer the question content for the real patient Pr represented by the question information output by the question reception function 113 (step S210). If it is determined in step S210 that the question content can be answered using the current patient digital twin, the missing information extraction function 1144 outputs the current question content to the digital twin answer function 1148 and proceeds to step S280.

On the other hand, if it is determined in step S210 that the question content cannot be answered using the current patient digital twin, the missing information extraction function 1144 extracts information and data (missing information) that are missing to answer the question content this time (step S220). The missing information extraction function 1144 then instructs the information acquisition function 112 to acquire the latest medical record data and examination data, and notifies the digital twin update function 1146 that there are information and data (missing information) that are missing to answer the current question content.

The processing circuitry 110 acquires the latest medical record data and examination data through the information acquisition function 112 (step S230). More specifically, the patient information acquisition function 1122 in the processing circuitry 110 acquires the latest medical record data, and the examination information acquisition function 1124 in the processing circuitry 110 acquires the latest examination data. Then, the information acquisition function 112 outputs the acquired latest medical record data and examination data to the digital twin processing function 114.

The processing circuitry 110 extracts differences (difference information) between the acquired latest medical record data and examination data and the medical record data and examination data stored in the information storage 120 through the digital twin processing function 114 (more specifically, the difference information extraction function 1142) (step S240). The difference information extraction function 1142 adds (stores) the difference information in the information acquisition function 112 and notifies the digital twin update function 1146 of the same.

When the difference information extraction function 1142 notifies the digital twin update function 1146 that the difference information has been added (stored) in the information acquisition function 112, the processing circuitry 110 updates the patient digital twin stored in the information acquisition function 112 on the basis of the latest medical record data and examination data stored in the information acquisition function 112 through the digital twin processing function 114 (more specifically, the digital twin update function 1146) (step S250). The digital twin update function 1146 stores the updated patient digital twin in the information acquisition function 112 and notifies the missing information extraction function 1144 of the same.

The processing circuitry 110 determines whether the updated patient digital twin stored in the information storage 120 can be used to answer the question content for the real patient Pr represented by the question information output by the question reception function 113 through the digital twin processing function 114 (more specifically, the missing information extraction function 1144) (step S260). If it is determined in step S260 that the question content can be answered using the updated patient digital twin, the missing information extraction function 1144 outputs the current question content to the digital twin answer function 1148 and proceeds to step S280.

On the other hand, if it is determined in step S260 that the question content cannot be answered using the updated patient digital twin, the missing information extraction function 1144 instructs the information acquisition function 112 to acquire the latest life log and personal data. Then, the processing circuitry 110 acquires the latest life log and personal data through the information acquisition function 112 (step S270). More specifically, the patient information acquisition function 1122 acquires the latest life log and personal data from the server device 30 (the life log storage device 322 and the personal data storage device 324 included in the patient information storage device 32) via the network NW. The information acquisition function 112 then outputs the acquired latest life log and personal data to the digital twin processing function 114, and the processing circuitry 110 returns the processing to step S240.

In the current processing of step S240, the processing circuitry 110 extracts differences (difference information) between the acquired latest life log and personal data and the life log and personal data stored in the information storage 120 through the digital twin processing function 114 (more specifically, the difference information extraction function 1142). The difference information extraction function 1142 then adds (stores) the difference information in the information acquisition function 112 and notifies the digital twin update function 1146 of the same.

When the difference information extraction function 1142 notifies the digital twin update function 1146 that the difference information has been added (stored) in the information acquisition function 112, in the current processing of step S250, the processing circuitry 110 re-updates the patient digital twin stored in the information acquisition function 112 on the basis of the latest life log and personal data stored in the information acquisition function 112 through the digital twin processing function 114 (more specifically, the digital twin update function 1146). The digital twin update function 1146 stores the re-updated patient digital twin in the information acquisition function 112 and notifies the missing information extraction function 1144 of the same.

In the current processing of step S260, the processing circuitry 110 determines whether it is possible to use the re-updated patient digital twin stored in the information storage 120 to answer the question content for the real patient Pr, represented by the question information output by the question reception function 113, through the digital twin processing function 114 (more specifically, the missing information extraction function 1144). In the current processing of step S260, if it is determined that the question content can be answered using the re-updated patient digital twin, the missing information extraction function 1144 outputs the current question content to the digital twin answer function 1148 and proceeds to step S280.

If it is determined in step S210 or step S220 that the question content can be answered using the current or updated (including re-updated) patient digital twin, the processing circuitry 110 answers the question content for the real patient Pr represented by the question information received by the question reception function 113 through the digital twin processing function 114 (more specifically, the digital twin answer function 1148) (step S280). Then, the processing circuitry 110 ends the processing of this flowchart in which the received question content for the real patient Pr is answered.

In this way, the processing circuitry 110 in the medical information processing device 100 updates the patient digital twin as necessary and answers and questions received for the real patient Pr. As a result, in the medical information processing device 100, the digital twin processing function 114 (more specifically, the digital twin update function 1146) in the processing circuitry 110 can use the latest state of the patient digital twin to answer questions for the real patient Pr received by the question reception function 113.

In the processing of the above-described flowchart, an example of a case in which the processing circuitry 110 updates the patient digital twin in two stages has been described. However, the patient digital twin may be updated in one stage. That is, if the processing circuitry 110 determines in the processing of step S210 that it is not possible to answer the question content using the current patient digital twin, the processing circuitry 110 may obtain the latest patient information and examination information (medical record data, examination data, life log, and personal data) and update the patient digital twin. The processing in this case may be equivalent to the processing of the above-described flowchart. Therefore, a detailed description of the processing of the processing circuitry 110 included in the medical information processing device 100 in this case will be omitted.

However, even if the patient digital twin is updated, it may not be possible to answer questions from the doctor Dr or the medical professional Dp. In this case, the processing circuitry 110 may output a question content to, for example, the notification control function 116, and notify the real patient Pr through the notification control function 116. As a result, if an answer is obtained from the real patient Pr, the doctor Dr or the medical professional Dp can obtain information and data regarding the content (question content) that they want to confirm when diagnosing even when the real patient Pr has not visited the hospital.

In this way, the medical information processing device 100 uses the patient digital twin to answer questions for the real patient Pr from medical professionals (doctor Dr or medical professional Dp) when diagnosing the disease that the real patient Pr is suffering from. Then, the medical information processing device 100 acquires patient information (which may include examination information) that records the living conditions of the real patient Pr, and updates the patient digital twin to a newer state. As a result, through the medical information processing device 100, the doctor Dr or the medical professional Dp can obtain information and data regarding the content (question content) that the doctor Dr or the medical professional Dp wants to confirm when diagnosing the disease that the real patient Pr is suffering from even when the real patient Pr has not visited the hospital, that is, when the doctor Dr or the medical professional Dp cannot directly ask the real patient Pr questions. Accordingly, the doctor Dr or the medical professional Dp who uses the medical information processing device 100 can more appropriately diagnose and examine the disease that the real patient Pr is suffering from to provide more appropriate treatment and medical procedures for the real patient Pr.

In the above description of the medical information processing device 100, it is assumed that there are matters that need to be reconfirmed with the real patient Pr after the real patient Pr who visited the hospital has gone home, or there are additional matters that need to be confirmed. However, it is also possible to ask questions to the medical information processing device 100, for example, before the real patient Pr visits the hospital.

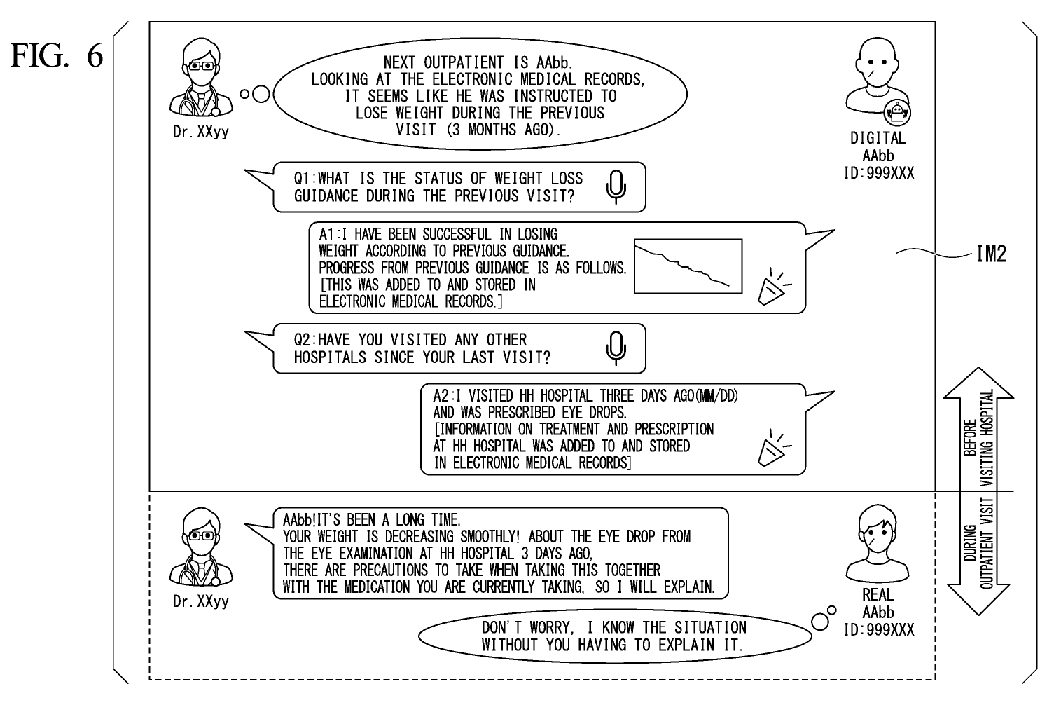

Here, an example of this case will be described. FIG. 6 is a diagram showing an example of questions to the medical information processing device 100 according to the embodiment and answers thereto. In FIG. 6, questions and answers are arranged in chronological order on a display screen IM2 when a display image generated by the display control function 115 is displayed on the display device. That is, the display screen IM2 shown in FIG. 6 shows more information on exchange between questions and answers than the range of the display image that can actually be displayed on the display device. Although exchange of questions and answers on the display screen IM2 shown in FIG. 6 is performed, for example, by voice input to a microphone and speech through a speaker, for ease of description, FIG. 6 shows an example in which voice is converted into text and displayed on the display screen IM2 by, for example, a voice recognition function in the large language model (LLM) provided in the patient digital twin. In actual display on the display device, a part of the range of the display image that can be displayed on the display device is displayed by moving sequentially from the top to the bottom of the paper. In other words, the questions and answers shown on the display screen IM2 are displayed by scrolling sequentially. Furthermore, the display screen IM2 shown in FIG. 6 schematically shows emotions of the doctor Dr (=XXyy) who asks questions and the real patient Pr (=AAbb) who visits the hospital as an outpatient.

The display screen IM2 shown in FIG. 6 shows an example of a case in which the next consultation is with real patient Pr (=AAbb), and thus the current status of “body weight loss guidance from three months ago” that was added (stored) in the electronic medical record by the medical information processing device 100 or that the doctor Dr gave directly to the real patient Pr, and “medical history since the previous visit” are confirmed before the real patient Pr visits the hospital. More specifically, the display screen IM2 shows an example of a case in which the doctor Dr first asks in natural language, “What is the status of the body weight loss guidance from the last visit?,” the digital patient Pd answers in natural language, “I succeeded in losing body weight as instructed last time,” and a notification indicating that data regarding this, “graph showing progress since the previous guidance,” has been added to and stored in the electronic medical record system 12 is given. In addition, the display screen IM2 shows an example of a case in which the doctor Dr subsequently asks in natural language, “Have you visited any other hospitals since your last visit?,” the digital patient Pd answers in natural language, “I visited HH Hospital three days ago (MM/DD) and was prescribed eye drops.”, and a notification indicating that information regarding this, “information regarding the treatment and prescription at HH Hospital,” has been added to and stored in the electronic medical record system 12 is given.

In addition, below the display screen IM2 shown in FIG. 6, an example of direct exchange between the doctor Dr and the real patient Pr on the basis of information and data obtained in advance by the medical information processing device 100 during outpatient visit of the real patient Pr to the hospital is shown. More specifically, an example of a case in which the doctor Dr informs the real patient Pr that he/she is aware that “body weight is decreasing steadily” on the basis of information that the real patient Pr has “successfully lost body weight,” and informs the patient of “precautions to take when using the medicine prescribed at the previous visit together with the eye drops prescribed at HH Hospital” on the basis of information regarding the “treatment and prescription at HH Hospital three days ago” is shown.

In this way, in the medical information processing device 100, the doctor Dr can ask questions before the real patient Pr visits the hospital to obtain answers in advance using the patient digital twin. Accordingly, the doctor Dr can make a more efficient diagnosis after the real patient Pr visits the hospital. This allows the real patient Pr to receive a consultation with peace of mind, with a high level of satisfaction, because the doctor Dr is aware of the current status.

In the above description of the medical information processing device 100, a case in which the real patient Pr can visit the hospital 10 has been described. However, there may be cases in which the real patient Pr is transported to the hospital 10 by emergency medical care, and the real patient Pr is unable to make a complaint. Even in this case, if the hospital 10 has the patient information and examination information of the real patient Pr (one or more pieces of medical record data, examination data, life log, and personal data), the medical information processing device 100 can answer questions from the doctor Dr (including the medical professional Dp) on behalf of the real patient Pr, and is therefore considered to be useful.

As described above, the medical information processing device of the embodiment uses the patient digital twin to answer questions for a patient that a medical professional wants to know when diagnosing the disease of the patient. In addition, the medical information processing device of the embodiment acquires patient information (which may include examination information) that records the living conditions of the patient and updates a patient digital twin to a newer state. As a result, the medical information processing device of the embodiment allows the medical professional to obtain information and data regarding the content (question content) that they want to know when diagnosing the disease of the patient even when the patient is not visiting the hospital (and they cannot ask the patient questions directly). This allows medical professionals using the medical information processing device of the embodiment to more appropriately diagnose and examine the disease of a patient and to provide more appropriate treatment and medical care for the patient.

Moreover, the medical information processing device of the embodiment answers questions for a patient from medical professionals other than doctors on behalf of the patient. That is, medical professionals other than doctors can obtain answers to questions for the patient without going through a doctor. This can reduce the burden not only on doctors and medical professionals other than doctors who use the medical information processing device of the embodiment, but also on patients.

Although an example of a case in which the medical information processing device is realized in one computer device or a server device on a network (not shown) has been described in the above embodiment, this is merely an example, and the medical information processing device or the functions realized by the medical information processing device may be realized by a configuration that combines a plurality of server devices or computer devices. The functional configuration, operation, and processing of the medical information processing device in this case may be equivalent to the functional configuration, operation, and processing of the medical information processing device in the above-described embodiment. Therefore, a detailed description of the medical information processing device in this case, or the functional configuration, operation, and processing that realize its functions, will be omitted.

The above-described embodiment can be represented as follows.

A medical information processing device that receives a question for a patient from a medical professional, and answers on behalf of the patient using a digital twin that reproduces a condition of the patient, the digital twin being an AI model trained by adding information on the patient to at least a large language model, the medical information processing device including processing circuitry,

wherein the processing circuitry is configured to:

receive the question;

acquire at least medical condition information representing a medical condition of the patient;

update the digital twin on the basis of the medical condition information;

use the digital twin to output answer information representing the answer to the received question; and

generate display data for presenting the answer information and cause a display device to display the display data.

According to at least one of the embodiments described above, a medical information processing device (100) that receives a question for a patient (real patient Pr) from a medical professional (Dr or Dp) and answers on behalf of the patient using a digital twin (patient digital twin) that reproduces a condition of the patient, the digital twin being an AI model trained by adding information on the patient to at least a large language model (LLM) includes processing circuitry that receives the question (113), acquires at least medical condition information (patient information and examination information) that represents a medical condition of the patient (112), updates the digital twin on the basis of the medical condition information (1146), uses the digital twin to output answer information representing the answer to the received question (1148), generates display data for presenting the answer information and causes a display device to display the display data (115), thereby realizing a medical information processing device, a medical information processing method, and a storage medium capable of supporting diagnoses and medical procedures by medical professionals by conveniently obtaining information about a patient that cannot be confirmed by main complaints of the patient or examination of a doctor.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing device that receives a question for a patient from a medical professional, and answers on behalf of the patient using a digital twin that reproduces a condition of the patient,
wherein the digital twin is an AI model trained by adding information on the patient to at least a large language model,
the medical information processing device comprising processing circuitry configured to:
receive the question;
acquire at least medical condition information representing a medical condition of the patient;
update the digital twin based on the medical condition information;
use the digital twin to output answer information representing the answer to the received question; and
generate display data for presenting the answer information and cause a display device to display the display data.

2. The medical information processing device according to claim 1,
wherein the processing circuitry is configured to:
determine whether the answer to the received question can be provided using the current digital twin;
extract missing information that is missing to answer the question if it is determined that the answer to the question cannot be provided;
acquire new medical condition information corresponding to the patient if the missing information is extracted; and
update the digital twin to include the new medical condition information.

3. The medical information processing device according to claim 2,
wherein the processing circuitry is configured to:
extract a difference between previous medical condition information of the patient and the new medical condition information of the patient; and
update the digital twin to include the new medical condition information if the difference between the previous medical condition information and the new medical condition information is extracted.

4. The medical information processing device according to claim 1, wherein the medical condition information includes patient information recorded when the patient was examined, and examination information representing results of examination performed on the patient.

5. The medical information processing device according to claim 4, wherein the patient information includes first patient information that is the patient information recorded when the patient was examined, and second patient information that is the patient information based on living records output by a collection device that collects living conditions of the patient.

6. The medical information processing device according to claim 5, wherein the second patient information includes third patient information about the patient that was not recorded when the patient was examined.

7. A medical information processing method, using a computer of a medical information processing device that receives a question for a patient from a medical professional, and answers on behalf of the patient using a digital twin that reproduces a condition of the patient, the digital twin being an AI model trained by adding information on the patient to at least a large language model, the medical information processing method comprising:
receiving, by the computer, the question;
acquiring, by the computer, at least medical condition information representing a medical condition of the patient;
updating, by the computer, the digital twin based on the medical condition information;
using, by the computer, the digital twin to output answer information representing the answer to the received question; and
generating, by the computer, display data for presenting the answer information and causing a display device to display the display data.

8. A non-transitory computer-readable storage medium storing a program for causing a computer of a medical information processing device that receives a question for a patient from a medical professional, and answers on behalf of the patient using a digital twin that reproduces a condition of the patient, the digital twin being an AI model trained by adding information on the patient to at least a large language model to:
receive the question;
acquire at least medical condition information representing a medical condition of the patient;
update the digital twin based on the medical condition information;
use the digital twin to output answer information representing the answer to the received question; and
generate display data for presenting the answer information and cause a display device to display the display data.
